(19) United States
(12) Patent Application Publication
(10) Pub. No.: US 2026/0237506 A1
(43) Pub. Date: Aug. 13, 2026

(54) SYSTEM AND METHOD FOR PROVIDING ONLINE MEDICAL TREATMENT

(71) Applicants: SEOUL NATIONAL UNIVERSITY HOSPITAL, Seoul (KR); HEALTHCONNECT CO., LTD., Seoul (KR)

(72) Inventors: Hoyoung LEE, Seongnam-si, Gyeonggi-do (KR); Eunyoung CHO, Seongnam-si, Gyeonggi-do (KR); Taeki KIM, Seongnam-si, Gyeonggi-do (KR); Hyunjung PARK, Seongnam-si, Gyeonggi-do (KR); Sujeong KIM, Seongnam-si, Gyeonggi-do (KR); Seunghyun JI, Seongnam-si, Gyeonggi-do (KR); Jungwon CHO, Seongnam-si, Gyeonggi-do (KR); Sanga KIM, Seongnam-si, Gyeonggi-do (KR); Hyeok KIM, Seoul (KR); Yongjun KWON, Seoul (KR); Jiae YOOK, Seoul (KR); Kyungseock YOO, Seoul (KR); Sanghyeung KIM, Seoul (KR)

(21) Appl. No.: 19/156,873

(22) PCT Filed: Feb. 15, 2024
(86) PCT No.: PCT/KR2024/095220

§ 371 Date: Aug. 14, 2025

(30) Foreign Application Priority Data
Feb. 15, 2023 (KR) 10-2023-0020245

(51) Int. Cl. G16H 40/67; G16H 10/60

(52) U.S. Cl. CPC G16H 40/67; G16H 10/60

(57) ABSTRACT

A system for providing online medical treatment according to one embodiment comprises: a medical personnel terminal which requests a server in an internal network of a hospital to open an online medical treatment room and invite other terminals including a patent terminal to the online medical treatment room; the patient terminal which receives a guidance message corresponding to the invitation from the server; and the server which transmits the guidance message to the patient terminal in response to the request and provides audio and/or video data, received from a terminal connected to the online medical processing room, to the other terminals connected to the online Medical treatment room, thereby providing an online medical treatment service.

### TECHNICAL FIELD

The disclosed embodiments relate to a system and a method for providing online medical treatment, and more particularly, to a technique for providing an online medical treatment service to a patient terminal and a medical personnel terminal.

### CROSS-REFERENCE TO RELATED APPLICATION

This application claims priority to Korean Patent Application No. 10-2023-0020245, filed on Feb. 15, 2023, the disclosure of which is incorporated herein by reference.

### BACKGROUND ART

In line with recent developments in IT technology, an online medical treatment service is disclosed. The online medical treatment service requires sharing and utilization of medical data and utilizes an electronic medical record (EMR) that computerized medical information of a patient who visits a hospital.

However, the EMR includes sensitive information that may infringe on privacy, such as personal information and medical history information. Although the online medical treatment service can be provided anytime, anywhere, there are concerns about privacy violations.

Thus, medical institutions should be able to provide convenient online-based medical services regardless of location while alleviating information anxiety. In addition, given that the medical service is directly related to life, it must be guaranteed to provide medical services at any time, regardless of time.

### DETAILED DESCRIPTION OF THE INVENTION

### Technical Solution

The disclosed embodiments are intended to provide a system and a method for providing online medical treatment.

### SUMMARY OF THE INVENTION

A system for providing online medical treatment according to an embodiment includes: a medical personnel terminal which requests a server in an internal network of a hospital to open an online medical treatment room and invite other terminals including a patent terminal to the online medical treatment room; the patient terminal which receives a guidance message corresponding to the invitation from the server; and the server which transmits the guidance message to the patient terminal in response to the request and provides audio and/or video data, received from a terminal connected to the online medical processing room, to other terminals connected to the online medical treatment room, thereby providing an online medical treatment service, wherein the patient terminal includes a terminal fixedly installed around a bed of the hospital assigned corresponding to the patient terminal and connected to the internal network through a WiFi-network of the hospital or a mobile-based terminal bypassing the internal network.

The medical personnel terminal may further request the server to invite at least one of the patient's guardian terminal and another medical personnel terminal.

The medical personnel terminal may request a search of the patient terminal by using patient information stored in a hospital database accessible through the internal network.

The server may include a first server and a second server and may be connected to the hospital database where an electronic medical record (EMR) of the hospital is stored through the first server or the second server.

The server may distribute and store data used and received for the online medical treatment service in a database for each server that provides the online medical treatment service.

The first server may store data transmitted and/or used by all terminals connected to a first online medical treatment room opened by the first server in a first database, and the second server may store data transmitted and/or used by all terminals connected to a second online medical treatment room opened by the second server in a second database.

The server may further include a switch that load-balances the request from the patient terminal to the first server or the second server.

The server of the internal network may use secure socket layer (SSL)-based sectional encryption when communicating with one or more terminals including the patient terminal.

The server may provide the guidance message to at least one of the medical personnel terminal, the patient terminal, the guardian terminal, and the other medical personnel terminal at a preset reservation date and time.

When the patient terminal is a mobile terminal, the guidance message may include a connection link, and when the patient terminal is a terminal fixedly installed around the hospital bed, the guidance message may be displayed on the patient terminal in a pop-up form.

A method for providing an online medical treatment according to an embodiment is performed by a system for providing online medical treatment including one or more processors, and memory for storing a program executed by the one or more processors, the method including: opening an online medical treatment room; inviting designated other terminals including a patient terminal to the online medical treatment room; sending a guidance message corresponding to the invitation; and providing audio and/or video data, received from a terminal connected to the online medical processing room, to the other terminals connected to the online medical treatment room, thereby providing an online medical treatment service, wherein the patient terminal includes a terminal that is fixedly installed around a bed of a hospital assigned corresponding to the patient terminal and is connected to an internal network through a WiFi-network of the hospital or a mobile-based terminal that bypasses the internal network.

The method may further include inviting at least one of the patient's guardian terminal and another medical personnel terminal.

The method may further include searching for the patient terminal using patient information stored in a hospital database accessible through the internal network.

The method may further include connecting, through the first server or the second server, to the hospital database storing the EMR of the hospital.

The method may further include distributing and storing data used and received for the online medical treatment service in a database for each server that provides the online medical treatment service.

The method may further include: storing, in a first database, data transmitted to the first server and/or used by all terminals connected to the first online medical treatment room opened by the first server; and storing, in a second database, data transmitted to the second server and/or used by all the terminals connected to the second online medical treatment room opened by the second server.

The method may further include load-balancing the request from the patient terminal to the first server or the second server using a switch included in the internal network and connected to the first server and the second server.

The method may further include communicating with one or more terminals including the patient terminal using the SSL-based sectional encryption.

The method may further include providing the guidance message to at least one of the medical personnel terminal, the patient terminal, the guardian terminal, and the other medical personnel terminal at a preset reservation date and time.

When the patient terminal is a mobile terminal, the guidance message may include a connection link, and when the patient terminal is a terminal installed around the hospital bed, the guidance message may be displayed on the patient terminal in a pop-up form.

### Effect of Invention

Disclosed embodiments may protect privacy of a patient's medical information by connecting to a server through an internal network.

The disclosed embodiments may ensure the stable online medical treatment service by connecting to a server with a duplexing path.

### BRIEF DESCRIPTION OF THE DRAWINGS

FIG. 1 is a block diagram of a system for providing online medical treatment according to an embodiment.

FIG. 2 is a flowchart of a process of providing an online medical treatment service.

FIG. 3 is a flowchart of a process of providing an online medical treatment service.

FIG. 4 is a diagram of a screen provided in a medical personnel terminal.

FIG. 5 is a diagram of a screen provided in the medical personnel terminal.

FIG. 6 is a diagram of a medical treatment screen where an online medical treatment service is provided.

FIG. 7 is a diagram of a screen showing the history of online medical treatment.

FIG. 8 is a diagram of a patient screen provided in a patient terminal.

FIG. 9 is a diagram of a patient screen provided in the patient terminal.

FIG. 10 is a flowchart of a method for providing online medical treatment according to an embodiment.

### BEST MODE FOR IMPLEMENTING THE INVENTION

Hereinafter, specific embodiments are described with reference to the drawings. The following detailed description is provided to facilitate a comprehensive understanding of a method, a device, and/or a system described herein. However, this is merely an example, and the present invention is not limited thereto.

In describing the embodiments, when it is determined that the detailed description of the known art related to the present invention may unnecessarily obscure the gist of the embodiment, the detailed description thereof will be omitted. In addition, the numerals (e.g., the first and the second) used in describing the embodiments are merely identifiers for distinguishing one component from another component. In addition, the terms described below, which are defined in consideration of the function in the present invention, may vary depending on the intention or practice of a user, an operator, or the like. Therefore, the definition should be made based on the content throughout this specification. The terms used herein are only for describing the embodiments and are not for limiting. Unless explicitly used otherwise, the singular forms include plural forms. In the present description, expressions, such as “comprising” or “including”, are intended to refer to certain components, numbers, steps, operations, elements, parts, or combinations thereof, and are not construed to exclude the presence or possibility of one or more other components, numbers, steps, operations, elements, parts, or combinations thereof.

Furthermore, embodiments described herein may have aspects that are entirely hardware, partially hardware and partially software, or entirely software. In this specification, a “unit”, a “module”, a “device”, a “server”, or a “system” refers to hardware, a combination of hardware and software, or a computer-related entity, such as software. For example, the unit, the module, the device, the server, or the system may refer to hardware constituting part or all of a platform and/or software, such as an application, for driving the hardware.

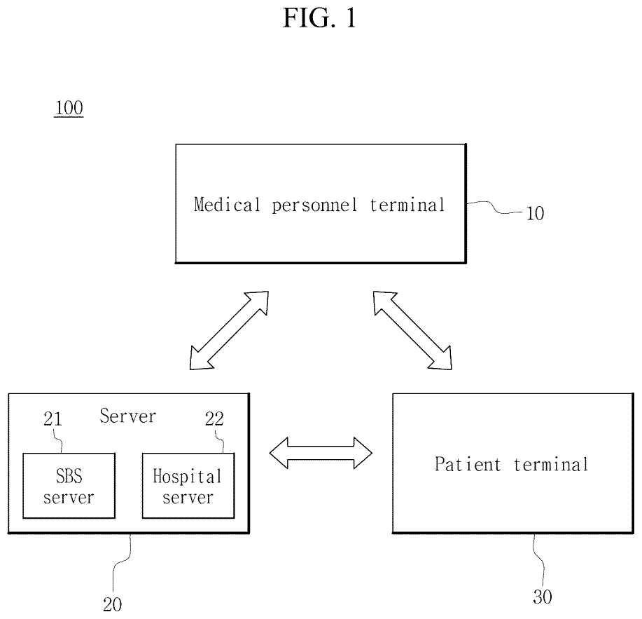

FIG. 1 is a block diagram of a system 100 for providing online medical treatment according to an embodiment.

Referring to FIG. 1, the system 100 for providing online medical treatment includes a medical personnel terminal 10, a server 20, and a patient terminal 30.

The system 100 for providing online medical treatment is a system for providing an online medical treatment service to a plurality of terminals. The online medical treatment service refers to a service that provides functions, such as a voice call, a video call, document sharing, screen sharing, and document sharing between the plurality of terminals through a virtual medical treatment room.

In particular, the system 100 for providing online medical treatment may be a system for providing medical services when the medical personnel terminal 10 and the patient terminal 30 participate in an online medical treatment room opened at the request of the medical personnel terminal 10.

The medical personnel terminal 10 is a terminal of medical personnel of a specific hospital. Specifically, the medical personnel terminal 10 may be a terminal used by the medical personnel who participate in the online medical treatment room.

The medical personnel terminal 10 may request the server 20 to open the online medical treatment room. The medical personnel terminal 10 may request the server 20 to invite the patient terminal corresponding to the designated patient to the online medical treatment room. The medical personnel terminal 10 may further request the server 20 to invite at least one of the patient's guardian terminal and another medical personnel terminal 10.

The medical personnel terminal 10, which is a general terminal, may include, for example, a desktop computer, a notebook computer, a tablet, a smartphone, and the like. The medical personnel terminal 10 may include any type of device capable of participating in the online medical treatment room.

The patient terminal 30 is a terminal of a patient in the same hospital as the medical personnel terminal 10. Specifically, the patient terminal 30 may be a terminal used by a patient participating in the online medical treatment room.

For example, the patient terminal 30, which is a general terminal, may include, for example, a desktop computer, a notebook computer, a tablet, a smartphone, or the like. The patient terminal 30 may include any type of device capable of participating in the online medical treatment room. The patient terminal 30 may bypass the internal network to be connected to the server 20.

As another example, the patient terminal 30 may include a smart bedside station (SBS)-based terminal fixedly installed around a bed of a hospital assigned to the patient. The SBS-based terminal may be a terminal that is fixedly installed around the bed of the hospital and connected to the server 20 through the internal network using a WiFi-network of the hospital.

The server 20 is a server 20 for providing the online medical treatment service. The server 20 may be built in the internal network to prevent leakage of medical information. The server 20 may include an SBS server 21 and a hospital server 22.

The SBS server 21 may be a server 20 for providing the online medical treatment service. The SBS server 21 may open the online medical treatment room in response to a request from the medical personnel terminal 10. The SBS server 21 may receive identification information of the medical personnel terminal 10 and the patient terminal 30 scheduled to participate in the online medical treatment room from the hospital server 22.

The SBS server 21 may receive the identification information of the patient terminal 30 corresponding to the patient information designated by the medical personnel terminal 10 from the hospital server 22 through an electronic medical record (EMR), and the SBS server 21 and the hospital server 22 may communicate in one direction.

The SBS server 21 may store identification information of the patient terminal 30 received from the hospital server 22. In this case, the identification information of the patient terminal 30 may include all kinds of information necessary for identifying the patient terminal 30, such as a telephone number or a personal identification number (PIN) of the patient terminal 30.

The SBS server 21 may distribute and store data transmitted and/or used by all terminals connected to the online medical treatment room. The SBS server 21 may include a first SBS server 21 and a second SBS server 21 to distribute and store the data transmitted and/or used by all terminals.

Specifically, the SBS server 21 may distribute and store data in a first database of the first SBS server 21. The SBS server 21 may distribute and store data in a second database of the second SBS server 21.

Specifically, the SBS server 21 may distribute and store data used and received for the online medical treatment service in a database for each server 20 that provides the online medical treatment service.

For example, when the first SBS server 21 provides the online medical treatment service, the first SBS server 21 may store data transmitted and/or used by all terminals connected to the first online medical treatment room opened by the first SBS server 21 in the first database.

For example, when the second SBS server 21 provides the online medical treatment service, the second SBS server 21 may store data transmitted and/or used by all terminals connected to the second online medical treatment room opened by the second SBS server 21 in the second database.

The first database and the second database are replicated with each other, so that unwritten information may be synchronized and updated.

The SBS server 21 may further include a switch for load-balancing to prevent load concentration. The SBS server 21 may perform load-balancing between the first SBS server 21 and the second SBS server 21 by using the switch for load-balancing.

For example, by using the switch, the SBS server 21 may load-balance the first online medical treatment room to be provided by the first SBS server 21 and the second online medical treatment room to be provided by the second SBS server 21.

The SBS server 21 enables the patient terminal 30 and the medical personnel terminal 10 to be connected to the online medical treatment room in response to the connection request of the patient terminal 30 and the medical personnel terminal 10 to the online medical processing room.

After confirming whether the patient terminal 30 and the medical personnel terminal 10 are authenticated, the SBS server 21 may allow connection to the online medical treatment room in response to the connection request of the patient terminal 30 and the medical personnel terminal 10.

The hospital server 22 may be a server 20 which receives personal information, medical information, and the like of the patient from the medical personnel terminal 10 and constructs a database of EMR.

The hospital server 22 may transmit the patient information to the medical personnel terminal 10 based on the EMR stored in the database in response to the information request of the medical personnel terminal 10 for the invitation to the online medical treatment room.

The patient information may include at least one of the patient's registration number, name, age, contact information, disease code, diagnosis name, and medical personnel.

The hospital server 22 may transmit, based on medical personnel information stored in the database, the medical personnel information to the medical personnel terminal 10 in response to the information request of the medical personnel terminal 10 for the invitation to the online medical treatment room.

The medical personnel information may include at least one of the medical personnel registration number, name, contact information, and medical department.

The medical personnel terminal 10, the server 20, and the patient terminal 30 may exchange data via a communication network. In particular, the medical personnel terminal 10, the server 20, and the patient terminal 30 preferably communicate through the internal network in order to prevent leakage of medical information.

The server 20 preferably communicates with one or more terminals including the patient terminal 30 by using secure socket layer (SSL)-based sectional encryption in order to prevent leakage of medical information.

In the disclosed embodiments, the medical personnel terminal 10, the server 20, and the patient terminal 30 may be implemented on a computing device including one or more processors and computer-readable recording media coupled to the processors. The computer-readable recording media may be internal or external to the processors and may be connected to the processors by various well-known means. The processors in the computing device may cause each computing device to operate in accordance with example embodiments described herein. For example, the processors may execute instructions stored in the computer-readable recording media. When executed by the processors, the computing device may perform operations according to example embodiments described herein.

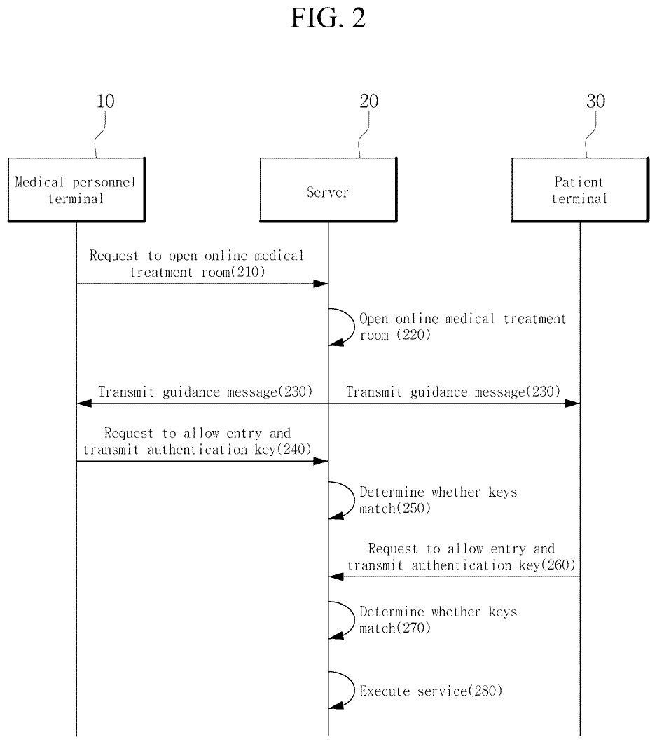

FIG. 2 is a flowchart of a process of providing an online medical treatment service.

In Step 210, the medical personnel terminal 10 requests the server 20 to open the online medical treatment room. The medical personnel terminal 10 may designate an opening reservation schedule and request the opening of the online medical treatment room together with invitation information. The invitation information may include information on terminals that may participate in the online medical treatment room.

In Step 220, the server 20 may open the online medical treatment room in response to the request of the medical personnel terminal 10.

In Step 230, the server 20 sends a guidance message corresponding to the invitation information. The guidance message may include a connection link to the online medical treatment room. The connection link may be transmitted to the terminal in the form of a text message (SMS) or a message push.

Specifically, when the patient terminal 30 is a mobile terminal, the guidance message including the connection link may be sent to the patient terminal 30. When the patient terminal is a terminal fixedly installed around the hospital bed, the guidance message may be displayed on the patient terminal in a pop-up form.

In Step 240, the medical personnel terminal 10 requests the server 20 to allow the ent to the online medical treatment room through the connection link. The medical personnel terminal 10 may request entry to the online medical treatment room while transmitting an authentication key. The medical personnel terminal 10 may request the server to allow the entry to the online medical treatment room by touching or clicking on the connection link through an input means including a mouse, a touchpad, or the like.

In Step 250, the server 20 compares the authentication key sent by the medical personnel terminal 10 with a pre-stored authentication key to confirm the connection authority of the medical personnel terminal 10. When it is determined that the authentication key matches the pre-stored authentication key, the server 20 may process the connection of the medical personnel terminal 10 to the online medical treatment room.

In Step 260, the patient terminal 30 requests the server 20 to allow the entry to the online medical treatment room through the connection link. The patient terminal 30 may request to allow the entry to the online medical treatment room while transmitting the authentication key. The patient terminal 30 may request entry to the online medical treatment room by touching or clicking on the connection link through an input means including a mouse, a touchpad, or the like.

In Step 270, the server 20 compares the authentication key sent by the patient terminal 30 with a pre-stored authentication key to confirm the connection authority of the patient terminal 30. When it is determined that the authentication key matches the pre-stored authentication key, the server 20 may process the connection of the patient terminal 30 to the online medical treatment room.

In Step 280, the server 20 executes the online medical treatment service when at least one of the patient terminal 30 and the medical personnel terminal 10 has entered. It is illustrated in Step 280 that the online medical treatment service is executed when both the patient terminal 30 and the medical personnel terminal 10 have entered, but it is only an example and is not limited thereto.

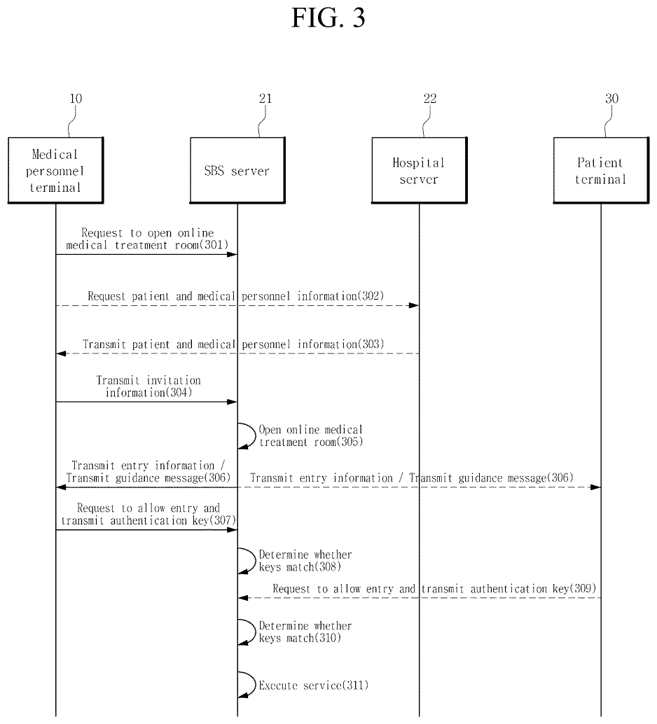

FIG. 3 is a flowchart of a process of providing the online medical treatment service.

Since Steps 307 to 311 are the same as Steps 240 to 280 in FIG. 2, descriptions of Step 307 to 311 that overlap with those of Steps 240 to 280 may be omitted.

In Step 301, the medical personnel terminal 10 may request the SBS server 21 to open online medical treatment room.

In Step 302, the medical personnel terminal 10 may request patient information and medical personnel information from the hospital server 22 to search for invitees. For example, the medical personnel terminal 10 may request at least one of patient information and medical personnel information by using an input means including a keyboard and a touch pad.

The medical personnel terminal 10 may directly request at least one of patient information and medical personnel information from the hospital server 22. Alternatively, the medical personnel terminal 10 may request at least one of patient information and medical personnel information from the hospital server 22 through the SBS server 21.

In Step 303, the hospital server 22 may transmit the patient information and medical personnel information to the medical personnel terminal 10 in response to the request of the medical personnel terminal 10 based on the EMR stored in the database. Alternatively, the hospital server 22 may transmit, to the SBS server 21, the patient information and medical personnel information to be provided to the medical personnel terminal 10 in response to the request of the medical personnel terminal 10.

In Step 304, the medical personnel terminal 10 may transmit invitation information including designated patient information and medical personnel information to the SBS server 21. The SBS server 21 may invite other terminals to the online medical treatment room based on the invitation information.

In Step 305, the SBS server 21 may open the online medical treatment room.

In Step 306, the SBS server 21 may send the guidance message to the patient terminal 30 corresponding to the patient information. Alternatively, the SBS server 21 may transmit the guidance message to the other medical personnel terminal 10 corresponding to the medical personnel information.

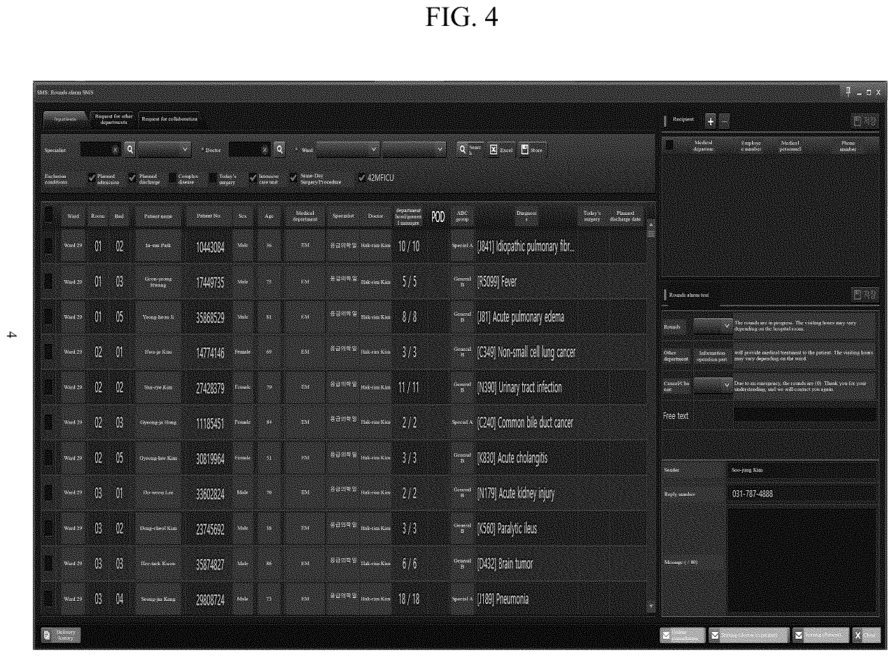

FIG. 4 is a diagram of a screen provided in the medical personnel terminal 10.

Referring to FIG. 4, the screen provided in the medical personnel terminal 10 includes a search screen for searching for an invitation target.

The medical personnel terminal 10 may request at least one of patient information and medical personnel information from the hospital server 22 to search for the invitation target.

The medical personnel terminal 10 may search for at least one of a ward, a hospital room, a hospital bed, a patient name, a medical department, a diagnosis name, a specialist in charge, and a doctor in charge, and may request at least one of patient information and medical personnel information from the hospital server 22.

The hospital server 22 may transmit the patient information including at least one of a ward, a hospital room, a hospital bed, a patient name, a patient number, a gender, an age, a medical department, a specialist in charge, a doctor in charge, a nurse in charge, a department head/general manager, a POD, an ABC group, a diagnosis name, whether surgery is performed today, a scheduled discharge date, whether hospitalization is scheduled, a complex disease, whether a patient is in an intensive care unit, a procedure center, and a surgery center, based on an the EMR stored in the hospital server 22 in response to the request of the medical personnel terminal 10.

The hospital server 22 may filter the selected items based on the input from the medical personnel terminal 10 and transmit only part of the patient information.

The medical personnel terminal 10 may designate part of the searched patient information and may request the SBS server 21 to invite the patient terminal 30 corresponding to the designated patient information and open the online medical treatment room.

The medical personnel terminal 10 may designate part of the searched patient information and may request the hospital server 22 to transmit the guidance message to the patient terminal 30 corresponding to the designated patient information. The guidance message may include a name and contact information of the medical personnel sending the guidance message, an online visit date and time, and whether the online visit is canceled or changed.

When a patient to be invited is selected by the medical personnel terminal 10, the hospital server 22 may switch from the search screen to an online medical treatment reservation screen that enables a request to the SBS server 21.

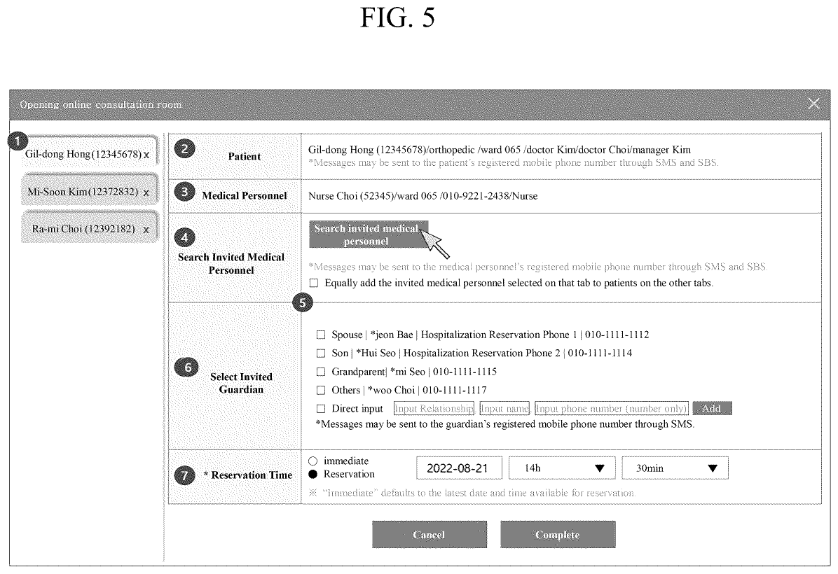

FIG. 5 is a diagram of a screen provided in the medical personnel terminal 10.

Referring to FIG. 5, the screen provided in the medical personnel terminal 10 includes the online medical treatment reservation screen.

The online medical treatment reservation screen of FIG. 5 may be a screen switched by the medical personnel terminal 10 clicking on the online consultation UI on the search screen of FIG. 4.

Referring to FIG. 5, the online medical treatment reservation screen may include at least one of a list tab, a first guest tab, a host tab, a second guest tab, a third guest tab, and a schedule tab.

The list tab includes a detail tab for identifying a designated patient through the search screen. In particular, when there are a plurality of patients designated through the search screen, the list tab may provide the detail tab to provide an online medical treatment reservation screen for each patient terminal 30.

In other words, the medical personnel terminal 10 may be provided with the online medical treatment reservation screen for each patient through the detail tab, and the online medical treatment reservation screen of each patient may be switched through the detail tab.

Although the medical treatment reservation screen for each patient is switched to an online medical treatment reservation screen of another patient through the detail tab, information written in each tab may be stored without being lost.

The first guest tab may provide the designated patient information through the search screen. The first guest tab may show part of the patient information from the hospital server 22. For example, the first guest tab may provide at least one of a patient name, a medical department, a ward, a specialist in charge, a doctor in charge, and a nurse in charge corresponding to the designated patient.

The host tab may provide medical personnel information of the medical personnel terminal 10 requesting to open the online medical treatment room. The medical personnel information may include medical personnel information input by the medical personnel terminal 10 or transmitted from the hospital server 22.

For example, the medical personnel information may include at least one of the medical personnel's name, ward, contact information, and nurse in charge.

The second guest tab may include a tab or an icon for the invitation to the online medical treatment room, other than the patient terminal 30. For example, the second guest tab may include a tab for directly receiving medical personnel information of the other medical personnel terminal 10 for the invitation to the online medical treatment room.

In another example, the second guest tab may generate a pop-up screen to search for t medical personnel upon clicking on the provided icon. The pop-up screen may be overwrapped and displayed on the online medical treatment reservation screen or may be switched from the online medical treatment reservation screen and displayed as a new screen.

The pop-up screen may include a search tab for inviting another medical personnel terminal 10. For example, the pop-up screen may include the search tab for inputting at least one of the medical department and the medical personnel's registration number and name.

The pop-up screen may output the medical personnel information corresponding to the searched word. The medical personnel information may be output as a list. The medical personnel terminal 10 may select at least one of the other medical personnel terminals 10 included in the list as the invitation target.

Then, the medical personnel information corresponding to the selected other medical personnel terminal 10 may be displayed on the second guest tab. For example, like the host tab, the second guest tab may display at least one of the medical personnel's name, medical department, contact information, and nurse in charge of the other medical personnel terminal 10.

The third guest tab may display information of the guardian terminal associated with the designated patient terminal 30 on the search screen. For example, the third guest tab may display at least one of the guardian's name, contact information, and relationship with the patient as information of the guardian terminal.

In other words, the third guest tab may be associated with the patient terminal 30 displayed on the first guest tab to display information of the guardian terminal. On the other hand, the third guest tab may further include a tab for directly inputting the guardian terminal associated with the patient terminal 30.

The schedule tab may include an input tab that causes the host to set an opening schedule of the online medical treatment room. For example, the schedule tab may include the input tab to set a schedule that is distinguished by at least one of a year, a month, a time, a minute, and a second.

The medical personnel terminal 10 may individually write an online medical treatment reservation screen for each patient through the list tab to collectively apply for an online medical treatment reservation.

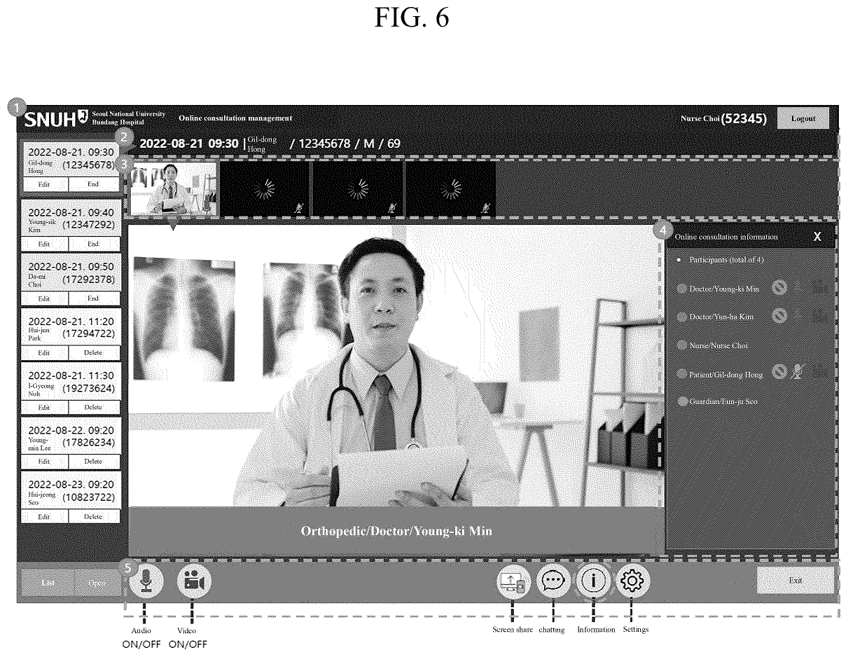

FIG. 6 is a diagram of a medical treatment screen where an online medical treatment service is provided.

Referring to FIG. 6, the medical treatment screen includes a list of online medical treatment rooms, information about the online medical treatment rooms, screens of the terminals entering the online medical treatment room, information about the terminals entering the online medical treatment room, and function buttons and list buttons that can be used in the online medical treatment room.

The online medical treatment list refers to a table in which information on each online medical treatment room opened by the medical personnel terminal 10 upon request to the SBS server 21 is recorded. For example, the online medical treatment list may display guest information(e.g., patient name, patient registration number) of each online medical treatment room, reservation date and time, whether each online medical treatment room is live, and the like.

The online medical treatment list may differently set a color indicating an online medical treatment room based on whether the online medical treatment room has ended, to identify whether each online medical treatment room is live.

The information on the online medical treatment room may include information on th online medical treatment room in which terminals enter. For example, the information on the online medical treatment room may include the patient information including the patient's name, age, registration number, and the reservation date and time.

The screen of each terminal entering the online medical treatment room may include an image captured by a camera of the terminal. At least one of the screens of the terminals entering the online medical treatment room may be selected through a user input and further output as a full screen. The full screen may further display information corresponding to an entity that is reading the full screen.

The information on each terminal entering the online medical treatment room may be output as a list of terminals currently entering the online medical processing room. For example, the information of each terminal may include at least one of a status (e.g., a doctor, a nurse, a patient, a guardian) and a name.

The function button may include a button that activates or deactivates at least one of audio, video, screen sharing, chatting, information, and setting functions. Each terminal may operate whether to activate the function by clicking the button through an input means.

The list button may be activated by input from the terminal. The activated list button may output a list of online medical treatment services used by the terminal. The list of the online medical treatment services may be classified and displayed in units of accounts in which the terminal is logged in.

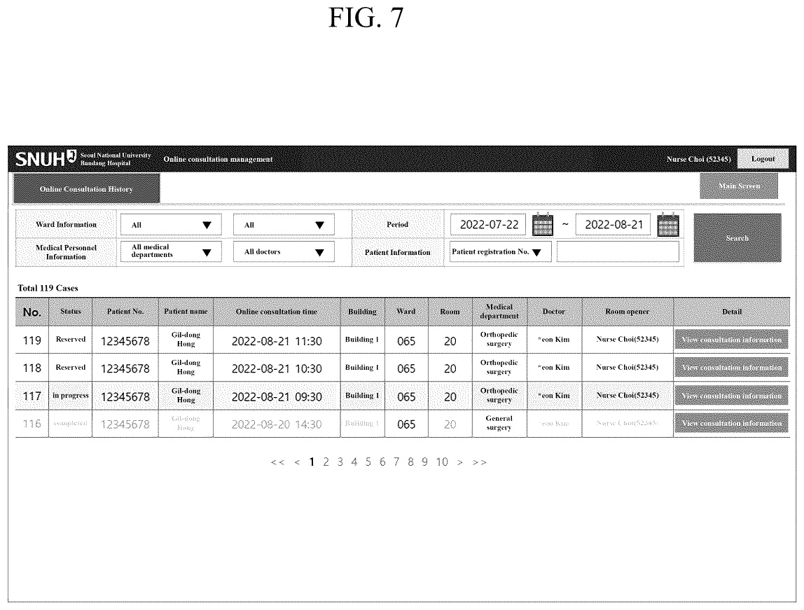

FIG. 7 is a diagram of a history screen of online medical treatment.

The history screen of FIG. 7 may be a screen switched by activating the list button of FIG. 6.

Referring to FIG. 7, the history screen includes a list of online medical treatment service The history screen may display information about the online medical treatment services provided from the history screen for a preset period from the switching request date.

For example, the history screen may display a status value that is distinguished by at least one of “reserved”, “in progress”, and “completed” for the online medical treatment room.

The history screen may display information on at least one of the patient number, the name, the online consultation time, the hospital building, the ward, the hospital room, the medical department, the specialist in charge, the room opener, the invited medical personnel, the invited guardian, and the consultation time of the patient participating in the online medical treatment room.

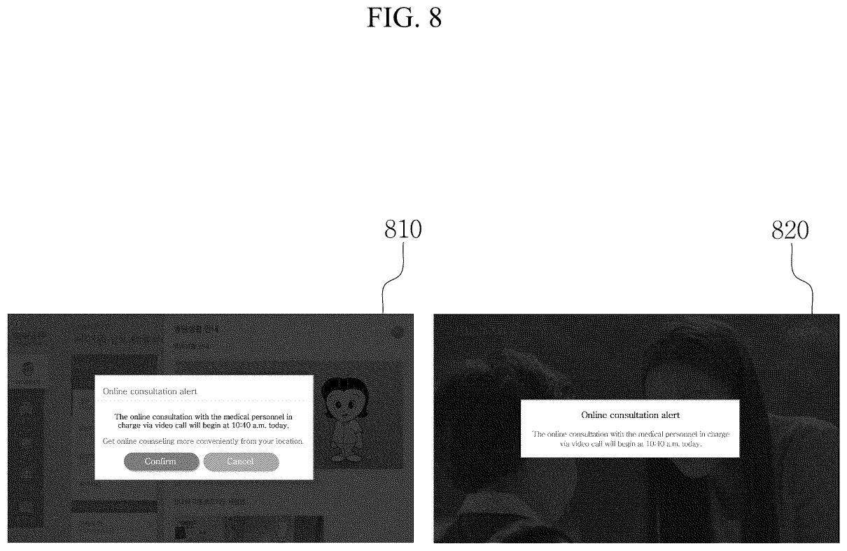

FIG. 8 is a diagram of a patient screen provided in the patient terminal 30. In particular, FIG. 8 assumes that the patient terminal 30 is an SBS-based terminal fixed to the hospital bed.

Referring to FIG. 8, the patient screen includes a first patient screen 810 and a second patient screen 820. The first patient screen 810 may be a screen on which the guidance message is displayed while a standby screen is shown.

The first patient screen 810 may include the guidance message indicated by an active pop-up message requesting a user response. When the patient terminal 30 is on standby, the pop-up message may be displayed on the patient screen to confirm reception of the patient.

The second patient screen 820 may be a screen on which a guidance message is displayed while an execution screen is shown. The second patient screen 820 may include a passive pop-up message that does not require a user response. The passive pop-up message may include a message that disappears without a separate response so as not to interfere with the current user's work.

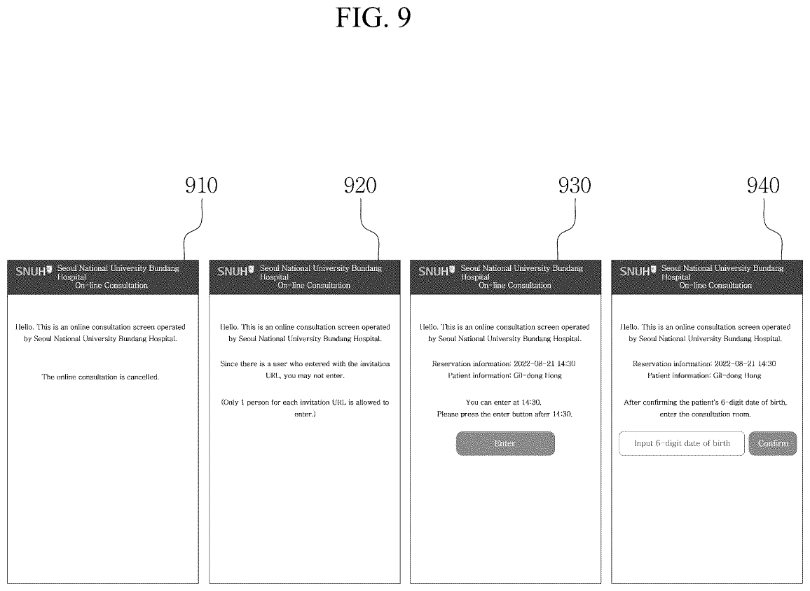

FIG. 9 is a diagram of a patient screen provided in the patient terminal 30.

Referring to FIG. 9, a patient screen provided in the patient terminal 30 may include a connection screen for guiding a connection result of the patient terminal 30 to the online medical treatment room.

The connection screen may be a screen displayed by clicking on the connection link included in the guidance message. The connection screen may be a screen provided to the patient terminal 30 when an authentication key corresponding to the online medical treatment room matches an authentication key transmitted by the patient terminal 30.

The first connection screen 910 may be a patient screen displayed when the patient terminal 30 enters the online medical treatment room where the opening request is deleted before the connection request of the patient terminal 30.

When there is another terminal already connected to the connection link, the second connection screen 920 may be a patient screen displayed for a terminal connected to the same link.

The third connection screen 930 may be a patient screen displayed on the patient terminal 30 when the connection process of the patient terminal 30 is performed before the reservation time.

The fourth connection screen 940 may be a patient screen displayed on the patient terminal 30 when the connection request of the patient terminal 30 is delivered after the reservation time. The patient terminal 30 may be connected to the patient terminal 30 by inputting an identification number (e.g., a PIN number) of the patient terminal 30.

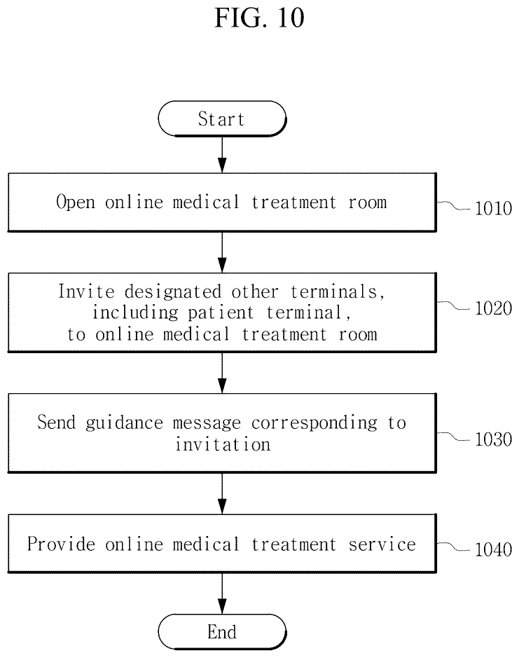

FIG. 10 is a flowchart of a method for providing online medical treatment according to an embodiment.

The method for providing online medical treatment according to an embodiment of FIG. 10 may be performed by the system for providing online medical processing 100 of FIG. 1.

Referring to FIG. 10, the system 100 for providing online medical treatment opens the online medical treatment room.

Then, the system 100 for providing online medical treatment invites designated other terminals including the patient terminal 30 to the online medical treatment room.

The system 100 for providing online medical treatment then sends the guidance message corresponding to the invitation. The system 100 for providing online medical treatment may transmit the guidance message to other terminals including the patient terminal 30.

Then, the system 100 for providing online medical treatment provides audio and/or video data received from the terminal connected to the online medical treatment room to other terminals connected to the online medical treatment room, thereby providing an online medical treatment service.

The patient terminal 30 may include a terminal that is fixedly installed around the hospital bed assigned corresponding to the patient terminal 30 and is connected to the internal network through the WiFi-network of the hospital or a mobile-based terminal that bypasses the internal network.

In the flowcharts shown in FIGS. 2, 3, and 10, the method is described by dividing the method into a plurality of steps, but at least some of the steps may be performed in a different order, may be performed together in combination with other steps, may be omitted, may be performed in sub-steps, or may be performed by adding one or more steps not shown.

An embodiment of the present invention may include a program for performing the methods described herein on a computer, and computer-readable recording media including the program. The computer-readable recording media may include program instructions, local data files, local data structures, and the like, alone or in combination. The media may be those specially designed and configured for the present invention, or those conventionally usable in the field of computer software. Examples of the computer-readable recording media include a magnetic medium, such as a hard disk, a floppy disk, and a magnetic tape, an optical recording medium, such as CD-ROM and a DVD, and a hardware device specifically configured to store and perform program instructions, such as ROM, RAM, flash memory, and the like. Examples of such programs may include machine language code made by a compiler, as well as advanced language code executable by a computer using an interpreter or the like.

While example embodiments of the present invention have been described above in detail, those skilled in the art may understand that various modifications can be made to the above-described embodiments without departing from the scope of the present invention. Therefore, the scope of the present invention should not be limited to the described embodiments, but should be defined by the following claims as well as those equivalent to the claims.

### INDUSTRIAL APPLICABILITY

A system and a method for providing online medical treatment according to an embodiment can be used in the digital medical industry because a medical personnel terminal and a patient terminal can be connected to a server built in an internal network of a hospital to receive an online medical treatment service.

1. A system for providing online medical treatment, the system comprising:
a medical personnel terminal which requests a server in an internal network of a hospital to open an online medical treatment room and invite other terminals including a patent terminal to the online medical treatment room;
the patient terminal which receives a guidance message corresponding to the invitation from the server; and
the server which transmits the guidance message to the patient terminal in response to the request and provides audio and/or video data, received from a terminal connected to an online medical treatment room, to the other terminals connected to the online medical treatment room, thereby providing an online medical treatment service, wherein the patient terminal comprises a terminal that is fixedly installed around a bed of the hospital assigned corresponding to the patient terminal and is connected to an internal network through a WiFi-network of the hospital, or a mobile-based terminal that bypasses the internal network.

2. The system of claim 1, wherein the medical personnel terminal further requests the server to invite at least one of the patient's guardian terminal and another medical personnel terminal.

3. The system of claim 1, wherein the medical personnel terminal requests search of the patient terminal by using patient information stored in a hospital database accessible through the internal network.

4. The system of claim 1, wherein the server comprises a first server and a second server and is connected to a hospital database where an electronic medical record (EMR) of the hospital is stored through the first server or the second server.

5. The system of claim 4, wherein the server distributes and stores data used and received for the online medical treatment service in a database for each server that provides the online medical treatment service.

6. The system of claim 5, wherein the first server stores data transmitted and/or used by all terminals connected to a first online medical treatment room opened by the first server in a first database, and
the second server stores data transmitted and/or used by all terminals connected to a second online medical treatment room opened by the second server in a second database.

7. The system of claim 4, wherein the server further comprises a switch for load-balancing a request from the patient terminal to the first server or the second server.

8. The system of claim 1, wherein the server of the internal network uses secure socket layer (SSL)-based sectional encryption when communicating with one or more terminals including the patient terminal.

9. The system of claim 2, wherein the server provides the guidance message to at least one of the medical personnel terminal, the patient terminal, the guardian terminal, and the other medical personnel terminal at a preset reservation date and time.

10. The system of claim 1, wherein, when the patient terminal is a mobile terminal, the guidance message includes a connection link, and when the patient terminal is a terminal fixedly installed around the hospital bed, the guidance message is displayed on the patient terminal in a pop-up form.

11. A method for providing online medical treatment, performed by a system for providing online medical treatment comprising one or more processors, and memory storing a program executed by the one or more processors, the method comprising:
opening an online medical treatment room;
inviting designated other terminals including a patient terminal to the online medical treatment room;
sending a guidance message corresponding to the invitation; and
providing audio and/or video data, received from a terminal connected to the online medical treatment room, to other terminals connected to the online medical treatment room, thereby providing an online medical treatment service,
wherein the patient terminal comprises a terminal that is fixedly installed around a bed of a hospital assigned corresponding to the patient terminal and is connected to an internal network through a WiFi-network of the hospital, or a mobile-based terminal that bypasses the internal network.

12. The method of claim 11, further comprising inviting at least one of the patient's guardian terminal and another medical personnel terminal.

13. The method of claim 11, further comprising searching for the patient terminal by using patient information stored in a hospital database accessible through the internal network.

14. The method of claim 11, further comprising connecting, via the first server or the second server, to a hospital database storing an electronic medical record (EMR) of the hospital.

15. The method of claim 14, further comprising distributing and storing data used and received for the online medical treatment service in a database for each server that provides the online medical treatment service.

16. The method of claim 15, further comprising storing, in a first database, data transmitted to the first server and/or used by all terminals connected to a first online medical treatment room opened by the first server, and storing, in a second database, data transmitted to the second server and/or used by all terminals connected to a second online medical treatment room opened by the second server.

17. The method of claim 14, further comprising load-balancing a request from the patient terminal to the first server or the second server using a switch included in the internal network and connected to the first server and the second server.

18. The method of claim 11, further comprising communicating with one or more terminals including the patient terminal by using secure socket layer (SSL)-based interval encryption.

19. The method of claim 12, further comprising providing the guidance message at a preset reservation date and time to at least one of the medical personnel terminal, the patient terminal, the guardian terminal, and the other medical personnel terminal.

20. The method of claim 11, wherein, when the patient terminal is a mobile terminal, the guidance message comprises a connection link, and when the patient terminal is a terminal installed around the hospital bed, the guidance message is displayed on the patient terminal in a pop-up form.
